(19) United States
(12) Patent Application Publication
(10) Pub. No.: US 2015/0284754 A1
(43) Pub. Date: Oct. 8, 2015

(54) METHODS AND APPARATUS RELATING TO LIQUEFACTION OF BIOMASS SLURRIES

(71) Applicant: ABENGOA BIOENERGY NEW TECHNOLOGIES, LLC, Chesterfield, MO (US)

(72) Inventors: Quang Nguyen, Chesterfield, MO (US); John Van Pelt, Denver, CO (US)

(21) Appl. No.: 14/437,366

(22) PCT Filed: Oct. 18, 2013
(86) PCT No.: PCT/US13/65559

§ 371 Date: Apr. 21, 2015

Related U.S. Application Data
(60) Provisional application No. 61/716,949, filed on Oct. 22, 2012.

(51) Int. Cl. C12P 19/02; C12M 1/00; C12P 7/10; C12P 19/14

(52) U.S. Cl. CPC C12P 19/02; C12P 19/14; C12M 21/12; C12P 7/10; C12M 45/06; C12P 2203/00; C12P 2201/00

(57) ABSTRACT

A liquefaction reactor includes multiple alternating plug flow and continuously-stirred regions. And, liquefaction of a biomass slurry in the reactor includes measuring pH of the biomass slurry in the reactor and, after each operation of measuring the pH, adjusting the pH of the biomass slurry in the reactor as needed to a value within a desired range. Liquefaction of the biomass slurry also includes adding enzymes to the biomass slurry in the reactor after measuring the pH of the biomass slurry in the reactor and adjusting the pH of the biomass slurry as needed. In addition, the pH of the biomass slurry may also be measured before the biomass slurry enters the reactor and, as needed, adjusted to a value within the desired range. Enzymes may then also be added to the biomass slurry before the biomass slurry enters the reactor.

### CROSS-REFERENCE TO RELATED APPLICATION

This application claims the benefit of, and priority to, U.S. Provisional Application No. 61/716,949, filed Oct. 22, 2012, the entire disclosure of which is incorporated herein by reference.

### FIELD

The present disclosure generally relates to biofuel production and, more specifically, to methods and apparatus for use in carrying out liquefaction of biomass slurries as a precursor to, for example, biofuel production (e.g., ethanol production, etc.).

### BACKGROUND

This section provides background information related to the present disclosure which is not necessarily prior art.

Lignocellulosic materials, such as wood, herbaceous material, agricultural residues, corn fiber, waste paper, pulp and paper mill residues, etc. as well as municipal solid waste can be used to produce biofuels. And typically, production of biofuels from such lignocellulosic material includes pretreatment (e.g., physical, chemical, etc.) of the lignocellulosic material to form a biomass slurry, liquefaction of the resulting biomass slurry, saccharification and fermentation of the liquefaction slurry, and then biofuel recovery (e.g., via distillation, etc.).

### SUMMARY

This section provides a general summary of the disclosure, and is not a comprehensive disclosure of its full scope or all of its features.

Example embodiments of the present disclosure generally relate to methods for carrying out liquefaction of slurries, for example, in liquefaction reactors, etc. using enzymes (e.g., cellulase, etc.). The liquefaction reactors may include alternating plug flow and continuously-stirred regions. In one example embodiment, a method for carrying out liquefaction of a biomass slurry generally includes measuring pH of the biomass slurry in multiple regions of a reactor; after each operation of measuring pH of the biomass slurry, adjusting the pH of the biomass slurry in the reactor as needed to a value within a desired range (e.g., between about 4 and about 6.5, etc.); and adding enzymes (e.g., cellulase enzymes, etc.) to the biomass slurry in the reactor. In some aspects of this example method, the enzymes are added to the biomass slurry in the reactor after at least two iterations of the operations of measuring pH of the biomass slurry and adjusting the pH of the biomass slurry as needed to a value within the desired range. In some aspects of this example method, the pH is measured in at least one plug flow region of the reactor. In some aspects of this example method, the pH is measured in at least one continuously-stirred region of the reactor. And, in some aspects of this example method, the pH is measured in multiple plug flow regions of the reactor.

In another example embodiment, a method for carrying out liquefaction of a pre-treated biomass slurry in a liquefaction reactor generally includes measuring an initial pH of the pre-treated biomass slurry (e.g., before the biomass slurry enters the reactor, etc.), and adjusting the initial pH of the biomass slurry as needed to a value within a desired range (e.g., between about 4 and about 6.5, between about 5.5 and about 6.5, etc.); adding enzymes (e.g., cellulase enzymes, etc.) to the pre-treated biomass slurry after adjusting the initial pH of the slurry to the value within the desired range; in a plug flow region of the liquefaction reactor, again measuring pH of the biomass slurry; and adjusting the pH of the biomass slurry measured in the plug flow region as needed to a value within the desired range.

Example embodiments of the present disclosure also generally relate to reactors for use in carrying out liquefaction of slurries. In one example embodiment, a tower reactor for use in carrying out liquefaction of a pre-treated biomass slurry generally includes alternating plug flow regions and continuously-stirred regions, agitators for moving the pre-treated biomass slurry in the continuously-stirred regions, probes positioned in multiple plug flow regions and configured to measure pH of the pre-treated biomass slurry in the multiple plug flow regions, first fluid lines positioned in communication with multiple ones of the continuously-stirred regions and configured to deliver an acid and/or a base to the pre-treated biomass slurry in the multiple ones of the continuously-stirred regions, and second fluid lines positioned in communication with multiple ones of the continuously-stirred regions and configured to deliver enzymes to the pre-treated biomass slurry in the multiple ones of the continuously-stirred regions.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a flow-chart illustrating an example embodiment of a method for carrying out liquefaction of a biomass slurry;

FIG. 2 is a schematic illustrating an example embodiment of an operation for carrying out liquefaction of a biomass slurry;

FIG. 3 is a schematic illustrating another example embodiment of an operation for carrying out liquefaction of a biomass slurry;

FIG. 4 is a schematic illustrating an example embodiment of a tower reactor for carrying out liquefaction of a biomass slurry; and

FIG. 5 is a schematic illustrating an example embodiment of an operation for producing biofuel from biomass material utilizing a liquefaction process of the present disclosure.

Corresponding reference numerals indicate corresponding parts throughout the several views of the drawings.

### DETAILED DESCRIPTION

Example embodiments will now be described more fully with reference to the accompanying drawings.

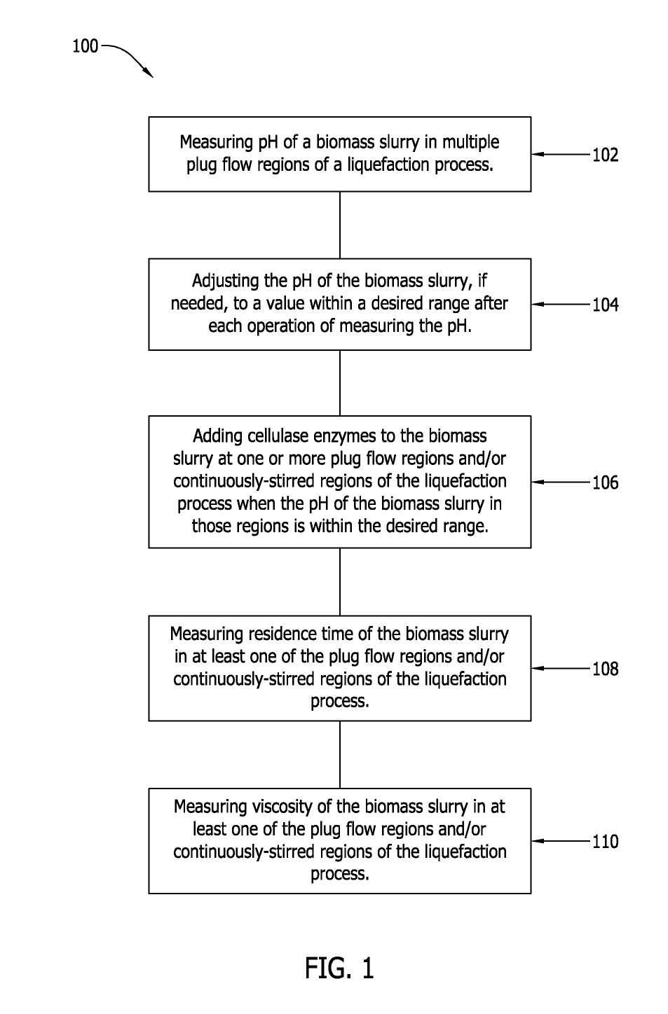

FIG. 1 illustrates an example method 100 for carrying out liquefaction of a biomass slurry. In the illustrated method, the biomass slurry is a cellulosic slurry formed by pre-treating lignocellulosic materials (e.g., wood, herbaceous material, agricultural residues, corn fiber, waste paper, pulp and paper mill residues, etc.). As such, the biomass slurry may also be referred to as a pre-treated biomass slurry, a cellulosic slurry, a pre-treated cellulosic slurry, etc. Suitable pre-treating operations can be used to form the biomass slurry including (without limitation) physical operations, chemical operations, and/or combinations thereof (e.g., grinding, heating, acid hydrolysis, steam explosion, combinations thereof, etc.). In other example embodiments, slurries (for use in liquefaction processes) may be formed from materials other than lignocellulosic materials (e.g., municipal solid waste, other similar biomass, etc.), and/or may be formed using other pre-treatment operations as desired and/or needed.

The liquefaction process of the illustrated method 100 is an enzymatic liquefaction process that utilizes enzymes to catalyze cellulolysis in the biomass slurry. In the illustrated method 100, cellulase enzymes are added to the biomass slurry to promote hydrolysis of cellulose in the biomass slurry (and conversion of the cellulose to glucose). With that said, it should be appreciated that suitable cellulase enzymes can be used in connection with the method 100 including, for example, enzymes from fungi, bacteria, protozoans, etc.; genetically engineered enzymes; etc. In other example embodiments, methods for carrying out liquefaction of biomass slurries may utilize enzymes other than cellulase enzymes, for example, enzyme cocktails, etc.

The cellulase enzymes used in the liquefaction process of the illustrated method 100 are pH sensitive and, for example, can significantly lose activity at pH values above about 6.5 or below about 4. As such, the illustrated method 100 is performed in connection with a series of multiple alternating plug flow and continuously-stirred regions (e.g., zones, containers, tanks, etc.). This allows for monitoring and/or controlling the pH of the biomass slurry throughout the series of plug flow and continuously-stirred regions (e.g., within each individual region, etc.). And in turn, the pH of the biomass slurry can be maintained within a desired range, for example, between about 4 and about 6.5, etc. throughout the liquefaction process to help promote efficient liquefaction (and cellulolysis) of the biomass slurry and help avoid denaturing of the cellulase enzymes. In one example embodiment, the cellulase enzymes used in the liquefaction process may have peak activity at a pH of about 5. As such, in this example embodiment, the pH of the biomass slurry may be maintained within a range of about 4.5 to about 5.5 (e.g., to retain greater than about ninety percent of the activity of the cellulase enzymes, etc.). In another example embodiment, different enzymes (e.g., enzymes other than cellulase enzymes, different types of cellulase enzymes, etc.) may be added to the biomass slurry at different locations in the liquefaction process as desired.

In the liquefaction process of the illustrated method 100, the continuously-stirred regions can make use of any suitable features (e.g., mechanical agitators, pumps, gravity, fluid recycle streams, other mixing means, etc.) to move, mix, stir, etc. the biomass slurry therein. What's more, in some example embodiments, the plug flow and continuously-stirred regions can be separate individual units arranged in series, while in other example embodiments they can all be located in series within a common reactor (e.g., a liquefaction reactor, a tower reactor, etc.), etc. Further, in some example embodiments, the plug flow and continuously-stirred regions can be oriented vertically (e.g., in towers, etc.), horizontally (e.g., with pumps, etc. moving the biomass slurry from one region to the next region, etc.), etc.

As shown in FIG. 1, the illustrated method 100 includes measuring the pH of the biomass slurry in multiple ones of the plug flow regions (e.g., in at least two of the plug flow regions, in less than all of the plug flow regions, in all of the plug flow regions, etc.) (as indicated generally at reference number 102 in FIG. 1). The pH measurements may be taken at any one or more desired location within the plug flow regions including, for example, locations where the biomass slurry moves into the plug flow regions, locations where the biomass slurry moves out of the plug flow regions, locations therebetween, etc. These measurements provide a check on the pH of the biomass slurry throughout the liquefaction process (e.g., throughout the series of plug flow and continuously-stirred regions, etc.), and can help ensure that the pH of the biomass slurry is maintained within a desired range (e.g., between about 4 and about 6.5, etc.) in each of the multiple plug flow and continuously-stirred regions (e.g., in preparation for adding cellulase enzymes to the biomass slurry, etc.). With that said, suitable devices including, for example, probes, colorimetric devices, chemical-based devices, pH measuring means, etc. can be used to measure the pH. In other example embodiments, pH measurements may be taken in only one of the plug flow regions in the liquefaction process. In addition, in some example embodiments pH measurements may be taken in one or more of the continuously-stirred regions of the liquefaction process.

The illustrated method 100 also includes adjusting the pH of the biomass slurry, if needed, to a value within the desired range (or to a specific desired value, etc.) after each operation of measuring the pH (as indicated generally at reference number 104 in FIG. 1). Where adjustment of the pH is needed, it can be accomplished by adding acid and/or base to the biomass slurry to raise or lower the pH. The acid and/or base can be added to the biomass slurry at any desired location in the liquefaction process (e.g., in any of the plug flow and/or continuously-stirred regions, etc.). For example, the acid and/or base may be added directly to the plug flow regions in which the corresponding pH measurements were taken (e.g., at locations in the plug flow regions immediately before the biomass slurry moves into following continuously-stirred regions, etc.), or to the continuously-stirred regions immediately following the plug flow regions in which the corresponding pH measurements were taken. Alternatively, if the measured pH of the biomass slurry at a location is already within the desired range, no adjustment would be required at that location. With that said, suitable devices including, for example, injection lines, injection nozzles, fluid lines, pumps, other fluid means, etc. can be used to add acid and/or base to the biomass slurry (e.g., within desired plug flow and/or continuously-stirred regions, etc.). Suitable acids may include, for example, sulfuric acid, hydrochloric acid, nitric acid, phosphoric acid, etc. And, suitable bases may include ammonia gas, aqua ammonia, sodium hydroxide, calcium hydroxide, potassium hydroxide, etc. In some example embodiments, the acid and/or base may be added directly to continuously-stirred regions in which corresponding pH measurements are taken.

The illustrated method 100 further includes adding cellulase enzymes to the biomass slurry at one or more regions (e.g., at one or more different plug flow regions, at one or more different continuously-stirred regions, etc.) through the liquefaction process. The cellulase enzymes may be added in conjunction with adjusting the pH of the biomass so that, for example, the cellulase enzymes are added to the biomass at one or more regions when the pH of the biomass slurry in those regions is within the desired range (as indicated generally at reference number 106 in FIG. 1). Or alternatively, the cellulase enzymes may be added in some regions through the liquefaction process independent of adjusting the pH of the biomass in those regions. The operation of adding cellulase enzymes to the biomass slurry may include, for example, adding the enzymes to the biomass slurry within one or more of the plug flow regions (e.g., at locations in the plug flow regions immediately before the biomass slurry moves into following continuously-stirred regions, etc.), adding the enzymes to the biomass slurry within one or more of the continuously-stirred regions, or adding the enzymes to the biomass slurry within combinations of the plug flow and the continuously-stirred regions. Suitable devices including, for example, injection lines, injection nozzles, fluid lines, pumps, other fluid means, etc. can be used to add the cellulase enzymes to the biomass slurry (e.g., within desired plug flow and/or continuously-stirred regions, etc.). Example enzyme dosages may include (without limitation) about 50 mg/g glucan in pretreated biomass solids or less (e.g., about 50 mg/g glucan, about 30 mg/g glucan, about 10 mg/g glucan, etc.).

In some aspects, the method 100 may also include measuring an initial pH of the biomass slurry before the biomass slurry enters, begins, etc. the liquefaction process. In these aspects, the method 100 may also include (although not required) adjusting the pH of the biomass slurry before it enters the liquefaction process, as needed, to a value within the desired range. And, the method 100 may then further include adding cellulase enzymes to the biomass slurry, again before the biomass slurry enters the liquefaction process (e.g., to initially reduce viscosity of the biomass slurry before it enters the liquefaction process to help improve mixing and thorough blending of the biomass slurry, pH control, enzyme activity, etc. during the liquefaction process, etc.). Here, it may also be desired to add a predetermined total amount of cellulase enzymes to the biomass slurry, taking into account the enzymes added before the biomass slurry enters the liquefaction process and the enzymes added during the liquefaction process. As such, a first portion of the predetermined total amount of enzymes (e.g., about sixty percent or less of the predetermined total amount of enzymes, etc.) may be added to the biomass slurry before it enters the liquefaction process, and then the remaining portion of the predetermined total amount of enzymes may be added to the biomass slurry during the liquefaction process (e.g., in one or more doses, etc.).

With continued reference to FIG. 1, the illustrated method 100 also includes measuring residence time of the biomass slurry in at least one of the plug flow and/or continuously-stirred regions (as indicated generally at reference number 108 in FIG. 1). For example, in the illustrated embodiment, the method 100 contemplates (while it is not required) measuring residence time of the biomass slurry in each of the plug flow and continuously-stirred regions (which may help in controlling viscosity of the biomass slurry as it proceeds through the liquefaction process). And as an example, the biomass slurry may be maintained in each of the plug flow regions for about five minutes or less and in each of the continuously-stirred regions for about two minutes or less. As such, in a liquefaction process utilizing five plug flow regions and five continuously-stirred regions, a total residence time for the liquefaction process may be about thirty-five minutes or less. In other example embodiments, methods may use different residence times in liquefaction process, for example, depending on biomass slurry sources, viscosity requirements, size requirements, etc.

Further, the illustrated method 100 includes measuring viscosity of the biomass slurry during the liquefaction process (e.g., in at least one of the plug flow regions and/or the continuously-stirred regions, etc.) (as indicated generally at reference number 110 in FIG. 1). As such, the viscosity of the biomass slurry can be monitored, tracked, etc. throughout the series of plug flow and continuously-stirred regions. This can include real-time rheological measurements (e.g., utilizing suitable instruments in each of the plug flow regions, etc.), or this can include removing samples of biomass slurry from each of the plug flow regions and analyzing the samples. In both cases, the monitoring allows for adjusting parameters of the liquefaction process, for example enzyme addition (e.g., quantity, rate, etc.), residence time, etc. to help ensure the biomass slurry is discharged from the liquefaction process at the desired viscosity. The monitoring may also allow for adjusting pre-treatment parameters (e.g., types of pre-treatment operations performed, etc.) used to form the biomass slurry. With that said (and without limitation), an example viscosity of the biomass slurry entering the liquefaction process may be about 20,000 centipoise or more (e.g., about 20,000 centipoise, about 30,000 centipoise, about 50,000 centipoise, etc.), and an example viscosity of the biomass slurry discharged from the liquefaction process may be about 8,000 centipoise or less (e.g., about 8,000 centipoise, about 5,000 centipoise, about 3,000 centipoise, etc.).

It should be appreciated that at least one or more of the operations of the illustrated method 100 may be performed automatically (e.g., via automated processes, etc.). As such, these at least one or more of the operations may be monitored and/or controlled remotely (e.g., at locations away from the liquefaction process, etc.).

In addition, in some example embodiments, methods for carrying out liquefaction of biomass slurries may include fewer operations than illustrated in FIG. 1, may include more operations than illustrated in FIG. 1, or may include operations other than those illustrated in FIG. 1 as needed. For example, in one example embodiment a method for carrying out liquefaction of a biomass slurry includes measuring a pH of the biomass slurry in a plug flow reactor, adjusting the pH of the biomass slurry to a value within a desired range, and adding cellulase enzymes to the biomass slurry once the pH is within the desired range. In this example embodiment, residence time and viscosity of the biomass slurry are not measured.

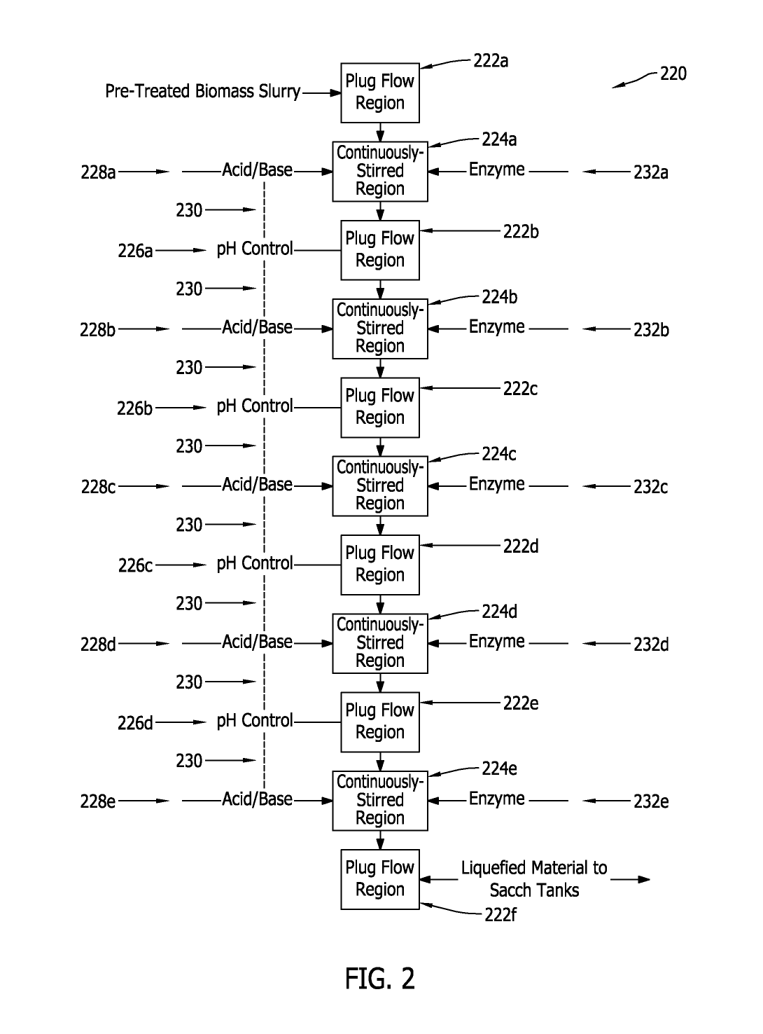

FIG. 2 illustrates an example operation 220 for carrying out liquefaction of a biomass slurry (e.g., a pre-treated biomass slurry, etc.) utilizing a series of alternating plug flow and continuously-stirred regions 222a-f and 224a-e, for example, in accordance with the method 100 previously described and illustrated in FIG. 1. The illustrated operation 220 utilizes (without limitation) six plug flow regions 222a-f and five continuously-stirred regions 224a-e. With that said, in other example embodiments, operations may utilize different numbers of plug flow regions and/or continuously-stirred regions depending, for example, on residence time constraints, viscosity requirements, enzyme requirements, size constraints, etc. What's more, it should be appreciated that the different plug flow and continuously-stirred regions 222a-f and 224a-e included in the illustrated operation 220 could each define an individual tank or reactor in the operation 220 (located in series), or could each be included together as part of a single tank or reactor (located in series) within the scope of the present disclosure.

In the illustrated operation 220, a pH of the biomass slurry is measured (as generally indicated at reference numbers 226a-d) in each of the four middle plug flow regions 222b-e to determine if the pH needs to be adjusted. If the measured pH is acceptable (e.g., within a desired range, for example, between about 4 and about 6.5, etc.; etc.), no further action is required. However, if the measured pH is not acceptable (e.g., outside the desired range, etc.), acid and/or base is added to the biomass slurry (as generally indicated at reference numbers 228a-e) to adjust the pH to an acceptable value (e.g., a value within the desired range, etc.). The illustrated operation 220 allows for adding acid and/or base to the biomass slurry in any one of the five continuously-stirred regions 224a-f and/or four middle plug flow regions 222b-e (as indicated by lines 230), when needed. As such, in some aspects of the operation 220, the acid and/or base can be added to the biomass slurry in the continuously-stirred region 224b-f immediately following the plug flow region 222b-e in which the corresponding pH measurement was taken.

The illustrated operation 220 also allows for adding enzymes (e.g., cellulase enzymes, different types of enzymes, etc.) to the biomass slurry in any one of the continuously-stirred regions 224a-f (as generally indicated at reference numbers 232a-e). As such, enzymes can be added as desired to any one or to multiple ones of the continuously-stirred regions 224a-f. As previously described, this may be done in conjunction with adjusting the pH of the biomass slurry so that, when adjusted, the pH is substantially optimized (e.g., within the desired pH range for the enzymes, etc.) to support the desired enzyme reactions (e.g., the cellulose hydrolysis, etc.). However, it should be appreciated that adding enzymes to any one or to multiple ones of the continuously-stirred regions 222a-f may alternatively be done, in one or more of the continuously-stirred regions 222a-f, independent of adjusting the pH of the biomass slurry in those regions.

In some aspects (and while not illustrated), the operation 220 may include measuring residence time of the biomass slurry in the plug flow regions 222a-f and/or continuously-stirred regions 224a-e, and/or may also include measuring viscosity of the biomass slurry during the liquefaction process, for example, in multiple ones of the plug flow regions 222a-f. This can help in monitoring progression, and effectiveness, of the liquefaction process.

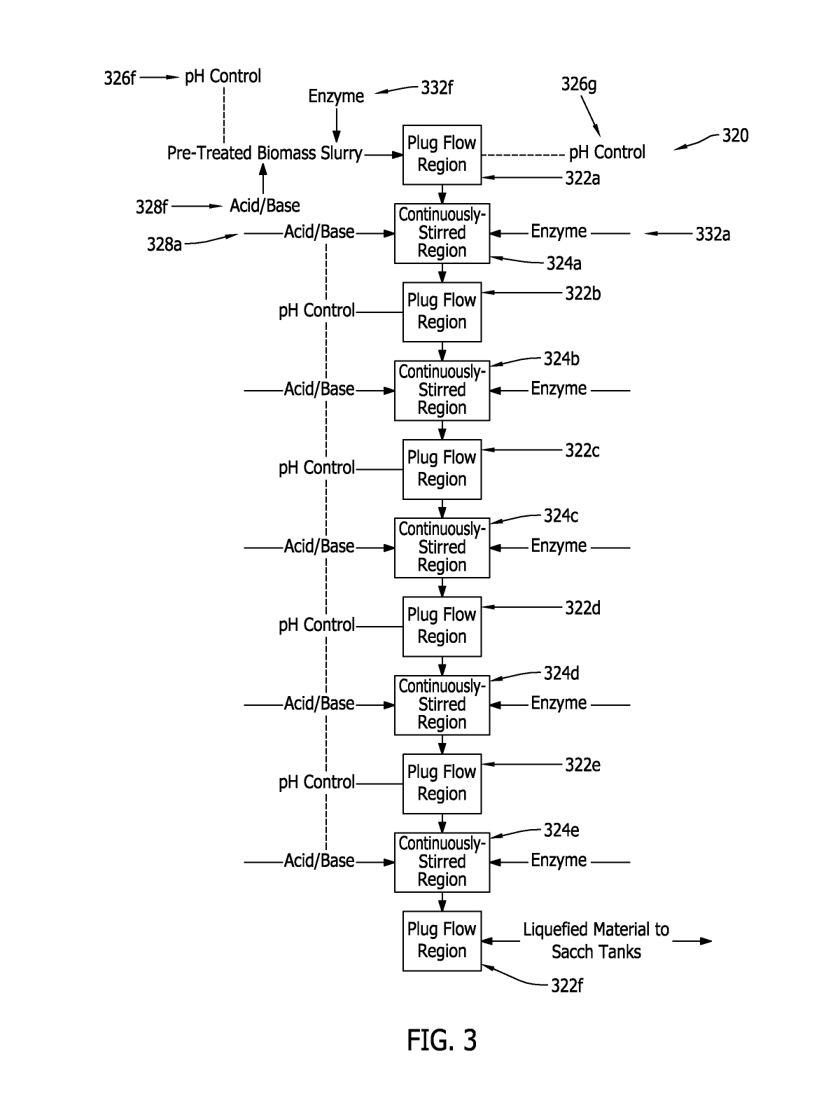

FIG. 3 illustrates another example operation 320 for carrying out liquefaction of a biomass slurry utilizing a series of alternating plug flow and continuously-stirred regions 322a-f and 324a-e, for example, in accordance with the method 100 previously described and illustrated in FIG. 1. The operation 320 of this embodiment is substantially similar to the operation 220 previously described and illustrated in FIG. 2. As such, the prior description of the operation 220 illustrated in FIG. 2 also generally applies to the instant operation 320 illustrated in FIG. 3.

With that said, in this embodiment, however, an initial pH of the biomass slurry is measured (as indicated generally at reference number 326f) before the biomass slurry enters (e.g., is pumped to, etc.) a first plug flow region 322a. In addition, the pH of the biomass slurry is also adjusted as needed (as indicated generally at reference number 328f) to fall within a desired range (e.g., between about 4 and about 6.5, etc.), and enzymes (e.g., cellulase enzymes, etc.) are then added to the biomass slurry (as indicated generally at reference number 332f) also before the biomass slurry enters the first plug flow region 322a. A pH of the biomass slurry can then be measured (as indicated generally at reference number 326g) in the first plug flow region 322a, with pH adjustment (as indicated generally at reference number 328a) and enzyme addition (as indicated generally at reference number 332a) following in a first continuously-stirred region 324a (with these operations then repeated as desired in the following plug flow and continuously-stirred regions 322b-e and 324b-e, for example, as described in connection with the operation 220 illustrated in FIG. 2, etc.).

Further in this embodiment, a portion of the total enzymes (e.g., about sixty percent or less, etc.) to be added to the biomass slurry as part of the operation 320 is added before the biomass slurry enters the first plug flow region 322a. The remaining portions of the enzymes are then added to the biomass slurry at one or more different ones of the subsequent continuously-stirred regions 324a-e (as indicated at reference numbers 232a-e).

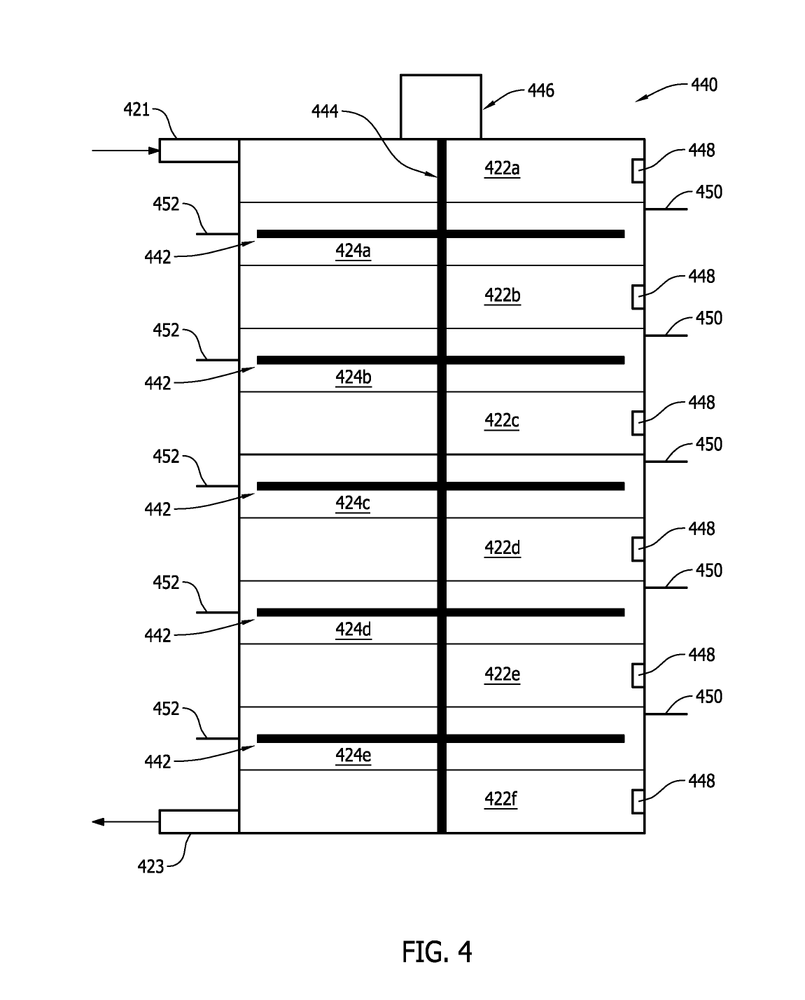

FIG. 4 illustrates an example tower reactor 440 (e.g., a liquefaction reactor, etc.) that can be used in carrying out liquefaction of a biomass slurry (e.g., in accordance with the method 100 previously described and illustrated in FIG. 1, with the operations 220 and 320 previously described and illustrated in FIGS. 2 and 3, etc.). The illustrated reactor 440 generally includes an inlet 421, six plug flow regions 422a-f, five continuously-stirred regions 424a-e. and an outlet 423. The pretreated biomass slurry enters the reactor 440 through the inlet 421 and exits the reactor through the outlet 423. And, the plug flow and continuously-stirred regions 422a-f and 424a-e are alternately arranged between the inlet 421 and the outlet 423 in a vertical orientation in the reactor 440 so that, in the liquefaction process, gravity can be used to move the biomass slurry downward through each of the regions 422a-f and 424a-e. In the illustrated embodiment, the inlet 421 is shown at the top of the reactor 440 and through a side of the reactor 440, and the outlet is shown at the bottom of the reactor 440 and through a side of the reactor 440. In other example embodiments, tower reactors may include inlets with entry points into the reactors through roofs or the reactors, and/or outlets with entry points into the reactors through floors of the reactors. In other example embodiments, tower reactors may alternatively be arranged to move (e.g., pump, etc.) biomass slurries upward through alternating plug flow and continuously-stirred regions in the tower reactors (e.g., such that inlets are toward bottom portions of the reactors and outlets are toward upper portions of the reactors, etc.).

In the illustrated embodiment, each of the continuously-stirred regions 424a-e of the reactor 440 includes an agitator 442 (e.g., a blade arrangement, etc.) configured to move (e.g., mix, etc.) the biomass slurry within a corresponding continuously-stirred region 424a-e of the reactor 440. Each agitator 442 is coupled along a common drive shaft 444 that extends through the reactor 440. And, a motor 446 is provided to rotate the drive shaft 444. As such, rotation of the common drive shaft 444 rotates each of the agitators 442, in turn moving (e.g., mixing, etc.) the biomass slurry in the corresponding continuously-stirred regions 424a-e. In other example embodiments, movement (e.g., mixing, etc.) of the biomass slurry may be achieved using individual agitators in each of the continuously-stirred regions, pumps, jets, recirculation streams, etc.

Also in the illustrated embodiment, each of the plug flow regions 422a-f includes a probe 448 configured to measure a pH of the biomass slurry in the corresponding plug flow region 422a-f. And, fluid lines 450 are located in each of the continuously-stirred regions 424a-e to deliver an acid and/or a base to the biomass slurry, as needed. As such, following measurement of the pH of the biomass slurry in each of the plug flow regions 422a-e, acid and/or base can be delivered to the biomass slurry in the following continuously-stirred region 424a-e, if needed, via the fluid lines 450.

Further in the illustrated embodiment, fluid lines 452 are located in each of the continuously-stirred regions 424a-e to deliver enzymes (e.g., cellulase enzymes, etc.) to the biomass slurry. Again, this can be done in conjunction with adjusting the pH of the biomass slurry so that the pH is substantially optimized through the reactor 440 to support the desired enzyme reactions (e.g., the cellulose hydrolysis, etc.). Or, alternatively, adding enzymes to the continuously-stirred regions 422a-e may be done, in one or more of the continuously-stirred regions 422a-e, independent of adjusting the pH of the biomass slurry in those regions.

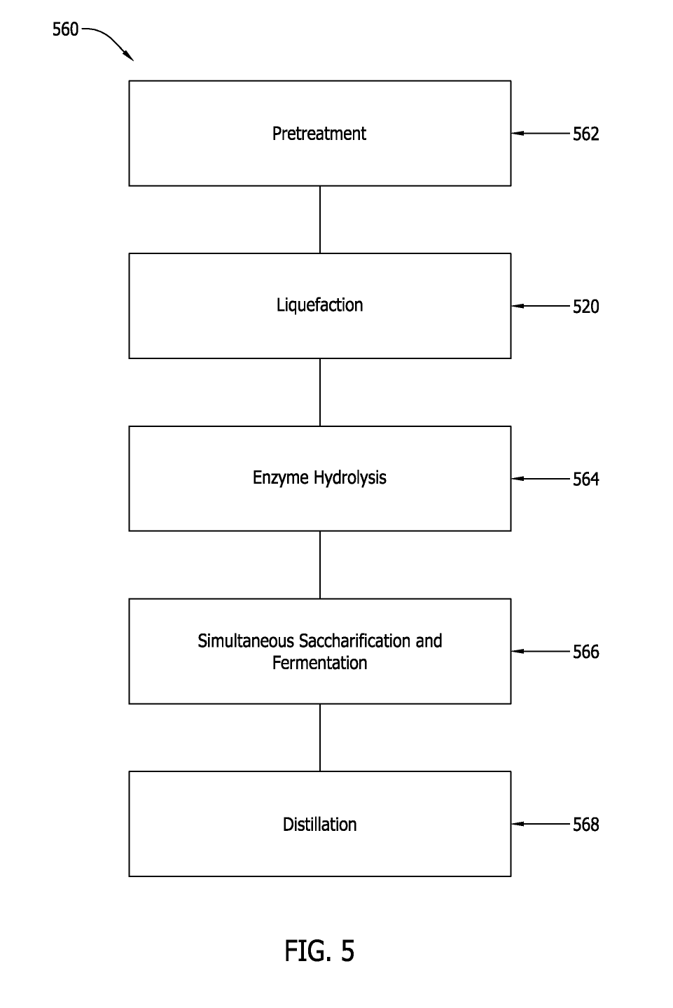

FIG. 5 illustrates a general operation 560 for producing biofuel (e.g., ethanol, etc.) utilizing a liquefaction process 520 of the present disclosure (e.g., in accordance with the method 100 previously described and illustrated in FIG. 1, in accordance with the operations 220 and 320 previously described and illustrated in FIGS. 2 and 3, in accordance with the tower reactor 440 previously described and illustrated in FIG. 4, etc.). The illustrated operation 560 utilizes biomass (e.g., wood, herbaceous material, agricultural residues, corn fiber, waste paper, pulp and paper mill residues, municipal solid waste, etc.) to produce the biofuel.

In the illustrated operation 560, the biomass is initially pretreated 562 using physical and/or chemical processes to form a biomass slurry (e.g., to liberate cellulose from the lignocellulosic material, etc.). Next, the pre-treated biomass slurry is subject to the liquefaction process 520 (e.g., hydrolysis of the liberated cellulose, etc.). Following the liquefaction process 520, the slurry is subject to an enzyme hydrolysis process 564, followed by a saccharification and fermentation process 566 (e.g., a separate saccharification process and a separate fermentation process, a simultaneous saccharification and fermentation (SSF) process, etc.). And then, a distillation process 568 is used to ultimately yield the biofuel.

Example embodiments are provided herein so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1.-11. (canceled)

12. A method for carrying out liquefaction of a pre-treated biomass slurry in a liquefaction reactor having multiple alternating plug flow and continuously-stirred regions, the method comprising:
measuring an initial pH of the biomass slurry before the biomass slurry enters the liquefaction reactor, and adjusting the initial pH as needed to a value within a desired range;
adding enzymes to the biomass slurry after adjusting the initial pH of the slurry to the value within the desired range and before the biomass slurry enters the liquefaction reactor;
in a plug flow region of the liquefaction reactor, measuring pH of the biomass slurry; and
in the liquefaction reactor, adjusting the pH of the biomass slurry measured in the plug flow region as needed to a value within the desired range.

13. The method of claim 12, wherein the desired pH range of the biomass slurry includes a pH range of between about 4 and about 6.5; and wherein adjusting the pH of the biomass slurry measured in the plug flow region as needed includes adding an acid and/or a base to the biomass slurry in the liquefaction reactor as needed to achieve the pH range of between about 4 and about 6.5;
further comprising adding enzymes to the biomass slurry in the liquefaction reactor after adjusting the pH of the biomass slurry measured in the plug flow region as needed to the value within the desired range.

14. The method of claim 13, wherein adding an acid and/or a base to the biomass slurry in the liquefaction reactor as needed includes adding the acid and/or the base to the biomass slurry as needed in a continuously-stirred region of the liquefaction reactor; and
wherein adding enzymes to the biomass slurry in the liquefaction reactor includes adding the enzymes to the biomass slurry in a continuously-stirred region of the liquefaction reactor.

15.-17. (canceled)

18. The method of claim 14, wherein a predetermined total amount of enzymes is added to the biomass slurry; wherein adding enzymes to the biomass slurry after adjusting the initial pH of the slurry to the value within the desired range and before the biomass slurry enters the liquefaction reactor comprises adding about sixty percent or less of the predetermined total amount of enzymes to the biomass slurry; and wherein adding enzymes to the biomass slurry in the liquefaction reactor after adjusting the pH of the biomass slurry measured in the plug flow region as needed to the value within the desired range comprises adding the remaining amount of the enzymes to the biomass slurry.

19. (canceled)

20. The method of claim 12, further comprising measuring viscosity of the biomass slurry in the liquefaction reactor.

21. The method of claim 12, further comprising pumping the biomass slurry into the liquefaction tower after the operations of measuring an initial pH of the biomass slurry before the biomass slurry enters the liquefaction reactor, adjusting the initial pH as needed to a value within a desired range, and adding enzymes to the biomass slurry after adjusting the initial pH of the slurry to the value within the desired range and before the biomass slurry enters the liquefaction reactor.

22. A tower reactor for use in carrying out liquefaction of a biomass slurry, the tower reactor comprising:
alternating plug flow regions and continuously-stirred regions;
agitators for moving the biomass slurry in the continuously-stirred regions;
probes positioned in multiple plug flow regions and configured to measure pH of the biomass slurry in the plug flow regions;
first fluid lines positioned in communication with multiple ones of the continuously-stirred regions and configured to deliver an acid and/or a base to the biomass slurry in the multiple ones of the continuously-stirred regions; and
second fluid lines positioned in communication with multiple ones of the continuously-stirred regions and configured to deliver enzymes to the biomass slurry in the multiple ones of the continuously-stirred regions.

23. The tower reactor of claim 22, further comprising instruments positioned in communication with multiple ones of the plug flow regions and configured to measure viscosity of the biomass slurry in the multiple ones of the plug flow regions.

24. A method for carrying out liquefaction of a biomass slurry in a reactor using enzymes, where the reactor includes alternating plug flow and continuously-stirred regions, the method comprising:
measuring pH of the biomass slurry in multiple regions of the reactor;
after each operation of measuring pH of the biomass slurry, adjusting the pH of the biomass slurry in the reactor as needed to a value within a desired range; and
adding enzymes to the biomass slurry in the reactor.

25. The method of claim 24, wherein measuring pH of the biomass slurry in multiple regions of the reactor includes measuring pH of the biomass slurry in multiple plug flow regions of the reactor.

26. The method of claim 24, wherein measuring pH of the biomass slurry in multiple regions of the reactor includes measuring pH of the biomass slurry in at least one continuously-stirred region of the reactor.

27. The method of claim 24, wherein adding enzymes to the biomass slurry in the reactor includes adding enzymes to the biomass slurry in the reactor after each of at least two iterations of the operations of measuring pH of the biomass slurry and adjusting the pH of the biomass slurry as needed to a value within a desired range

28. The method of claim 24, wherein each operation of adjusting the pH of the biomass slurry in the reactor as needed to a value within a desired range includes adding an acid and/or a base to the biomass slurry as needed.

29. The method of claim 28, wherein adding an acid and/or a base to the biomass slurry as needed includes adding the acid and/or the base to the biomass slurry as needed in a continuously-stirred region of the reactor.

30. The method of claim 24, wherein adjusting the pH of the biomass slurry in the reactor as needed to a value within a desired range includes adjusting the pH of the biomass slurry in the reactor as needed to a value between about 4 and about 6.5.

31. The method of claim 24, wherein adding enzymes to the biomass slurry in the reactor includes adding the enzymes to the biomass slurry in a continuously-stirred region of the reactor.

32. (canceled)

33. The method of claim 24, further comprising measuring viscosity of the biomass slurry in at least one plug flow region of the reactor.

34. The method of claim 33, further comprising discharging the biomass slurry from the liquefaction tower with a viscosity of about 8,000 centipoise or less.

35. The method of claim 24, further comprising retaining the biomass slurry in each plug flow region of the reactor for about five minutes or less and retaining the biomass slurry in each continuously-stirred region of the reactor for about two minute or less.

36. The method of claim 24, wherein the biomass slurry includes lignocellulosic material; and wherein the enzymes include cellulase enzymes;
further comprising:
pre-treating the lignocellulosic material using physical and/or chemical processes to liberate cellulose from the lignocellulosic material and form the biomass slurry;
introducing the biomass slurry into the reactor; and
saccharifying the biomass slurry to produce a biofuel, after performing the operations of measuring pH of the biomass slurry in multiple regions of the reactor, adjusting the pH of the biomass slurry in the reactor and adding enzymes to the biomass slurry in the reactor.
